(19) United States
(12) Patent Application Publication
(10) Pub. No.: US 2004/0022685 A1
(43) Pub. Date: Feb. 5, 2004

(54) Identification apparatus

(76) Inventors: Kirat Singh, Manchester (GB); Nicholas John Goddard, Manchester (GB); Peter Robert Fielden, Manchester (GB); Stephan Mohr, Manchester (GB)

Correspondence Address: John H Allie, Woodard Emhardt Moriarty McNett & Henry, Bank One Center/Tower, 111 Monument Circle, Suite 3700, Indianapolis, IN, 46204-5137, US

(21) Appl. No.: 10/381,170

(22) PCT Filed: Sep. 20, 2001
(86) PCT No.: PCT/GB01/04200

(30) Foreign Application Priority Data
Sep. 20, 2000 (GB) 0023041.7

(51) Int. Cl. G01N 33/00

(52) U.S. Cl. 422/82.08; 422/73

(57) ABSTRACT

An identification apparatus comprising a chamber having one or more buffer fluid inlets and a plurality of sample fluid inlets, the chamber being configured such that the buffer fluid focuses the sample fluid into sample fluid streams, the sample fluid streams and buffer fluid being contained in a single laminar flow, wherein the apparatus is provided with means for directing optical excitation simultaneously at a plurality of the sample fluid streams to allow parallel fluorescence and/or scattering measurements.

The present invention relates to an identification apparatus, and particularly though not exclusively to an identification and sorting apparatus of the type commonly referred to as a flow cytometer.

A flow cytometer provides optical identification and separation of cells and/or metaphase chromosomes based on light scattering and fluorescence.

In a known flow cytometer, a sample is focussed into a sample stream which is surrounded by sheath fluid. A focussed laser beam is directed perpendicularly across the sample stream, and induces fluorescence of fluorescent sample particles.

A flow cytometer can be used either as an analytical tool, counting the number of fluorescent labelled cells in a population, or to separate labelled and unlabelled cells for subsequent growth of the labelled cells population.

Recently, flow cytometers have been fabricated on a very small scale using microchip technology. PCT/US99/13542 describes a flow cytometer of this type, in which sample cells are driven through the flow cytometer using electromotive means. The flow cytometer described in PCT/US99/13542 includes a sample which is focussed into a stream of cells by buffer fluid. The stream of cells is directed either towards a sample outlet or towards a waste outlet, by altering an electric potential applied to a sample collection reservoir that is in fluidic connection with the sample outlet.

An advantage of flow cytometers is that they look at large numbers of individual cells, and makes possible the separation of populations with, for example, particular surface properties.

A disadvantage of known flow cytometers is that they are only capable of identifying and sorting a single flow of cells.

It is an object of the present invention to provide an identification apparatus which overcomes the above disadvantage.

According to a first aspect of the invention there is provided an identification apparatus comprising a chamber having one or more buffer fluid inlets and a plurality of sample fluid inlets, the chamber being configured such that the buffer fluid focuses the sample fluid into sample fluid streams, the sample fluid streams and buffer fluid being contained in a single laminar flow, wherein the apparatus is provided with means for directing optical excitation simultaneously at a plurality of the sample fluid streams to allow parallel fluorescence and/or scattering measurements.

Suitably, the chamber supports an optical mode centred on the single laminar flow, which passes through the sample fluid streams, thereby providing the optical excitation.

Suitably, the optical mode propagates across the single laminar flow in a direction substantially perpendicular to the direction of flow.

Suitably, the optical mode propagates along the single laminar flow in a direction substantially parallel to the direction of flow.

Suitably, the chamber is defined by upper and lower walls having refractive indices greater than the refractive index of the buffer fluid and sample fluid, and the optical mode is a leaky waveguide mode.

Suitably, the upper and lower walls are constructed from a polymer.

Suitably, the leaky waveguide mode is a light condenser mode.

Suitably, the optical excitation directing means comprises a diffracting beam splitter for separating a laser beam into a plurality of beams, and means for directing the plurality of beams at the sample fluid streams

Suitably, the sample fluid inlets are located downstream of at least some of the one or more buffer fluid inlets

Suitably, the sample fluid contains fluorescent labelled cells, the optical excitation has a wavelength suitable for exciting fluorescence of the fluorescent labelled cells, and the apparatus is provided with a detector to detect fluorescence of the fluorescent labelled cells.

Suitably, the detector is an array, each channel of the array detecting fluorescence from a different sample fluid stream.

Suitably, the apparatus is provided with an imaging spectrograph arranged to image fluorescence from sample fluid streams, and the array is a two-dimensional array arranged to detect different wavelengths of fluorescence emission.

Suitably, the apparatus is provided with a collection outlet and a waste outlet, and a sample stream is directed to the collection outlet in response to the detection of a fluorescent labelled cell in that sample fluid stream.

Suitably, a plate is located over the collection outlet and waste outlet, the plate having a collection opening and a: waste opening, wherein when the plate is in a waste position the waste opening and the waste outlet are substantially aligned and the collection opening and the collection outlet are substantially misaligned to allow a lesser amount of fluid to pass into the collection outlet, and when the plate is in a collection position the collection opening and collection outlet are substantially aligned and the waste opening and waste outlet are substantially misaligned.

Suitably, the collection opening and the waste opening are arranged such that the total flow of fluid into the collection outlet and waste outlet is substantially unchanged by movement of the plate from the collection position to the waste position.

Suitably, the openings and the holes are substantially circular, and are arranged to have an overlapping area of at least 20% when they are substantially misaligned.

Suitably, the chamber is provided with a plurality of separator walls having tapered upstream ends, the walls being located downstream of the buffer fluid inlet, and being arranged to separate the buffer fluid into a plurality of flows.

Suitably, a sample inlet is located substantially immediately downstream of each separator wall, such that the flow of buffer fluid around the sample inlet hydrodynamically focuses the sample fluid into a sample fluid stream.

Suitably, the sample fluid inlets and at least some of the buffer fluid inlets open onto a substantially planar surface, the inlet openings lying in substantially the same plane.

Suitably, at least some of the buffer fluid inlets are located downstream of the sample fluid inlets, thereby providing a layer of buffer fluid between the sample fluid streams and the substantially planar surface.

Suitably, the sample fluid inlets comprise fingers extending in a downstream direction, each finger being provided with a channel which carries the sample fluid to an opening at a downstream end of the finger.

Suitably, each finger is spaced way from upper and lower surfaces of the chamber, such that the buffer fluid flows along sides of the finger, and hydrodynamically focuses sample fluid emitted from the finger into a sample fluid stream.

Suitably, the fingers are constructed from stainless steel.

Suitably, the identification apparatus is fabricated by injection moulding a plurality of layers and then bonding those layers together.

According to a second aspect of the invention there is provided an identification apparatus comprising a chamber having one or more buffer fluid inlets and one or more sample fluid inlets, the chamber being configured such that the buffer fluid focuses the sample fluid into one or more sample fluid streams, wherein the apparatus is provided with a collection outlet and a waste outlet in separated portions of the chamber, and means for directing the sample fluid stream towards the collection outlet as required by directly heating buffer fluid in a portion of the chamber which is located upstream of the collection outlet so as to modify the fluid properties of the buffer fluid.

Suitably, the buffer fluid is directly heated using a laser.

Suitably, the buffer fluid contains a dye to provide enhanced absorption of the laser.

Suitably, the chamber is provided with electrodes which are in contact with the buffer fluid, and the buffer fluid is directly heated by passing a current between the electrodes via the buffer fluid.

Suitably, the current passed between the electrodes is alternated to avoid significant electrolysis and gas bubble generation at the electrodes.

Suitably, the buffer fluid includes a viscosity modifying substance to provide enhanced changes of the viscosity of the buffer fluid in relation to changes of fluid temperature.

Suitably, the viscosity modifying substance is glycerol or a polymer.

The second aspect of the invention may include any suitable feature of the first aspect of the invention.

A specific embodiment of the invention will now be described by way of example only, with reference to the accompanying figures in which:

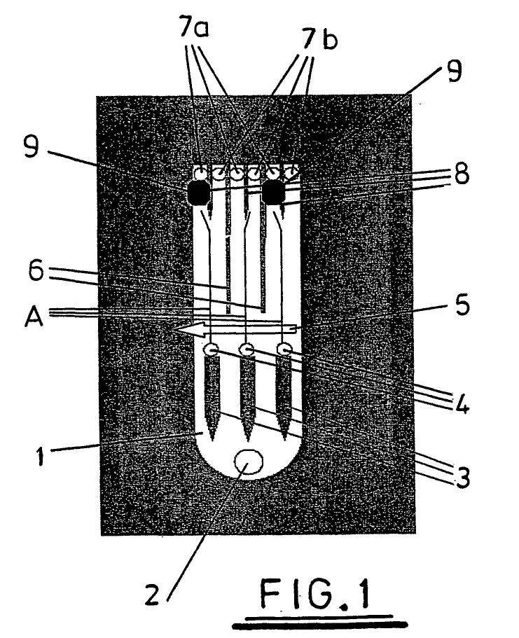

FIG. 1 is a schematic illustration of a flow cytometer which embodies the invention;

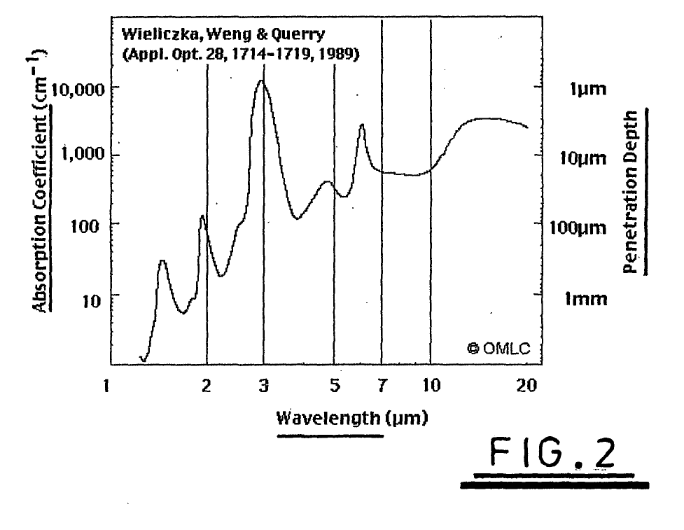

FIG. 2 is a graph illustrating the absorption coefficient of water;

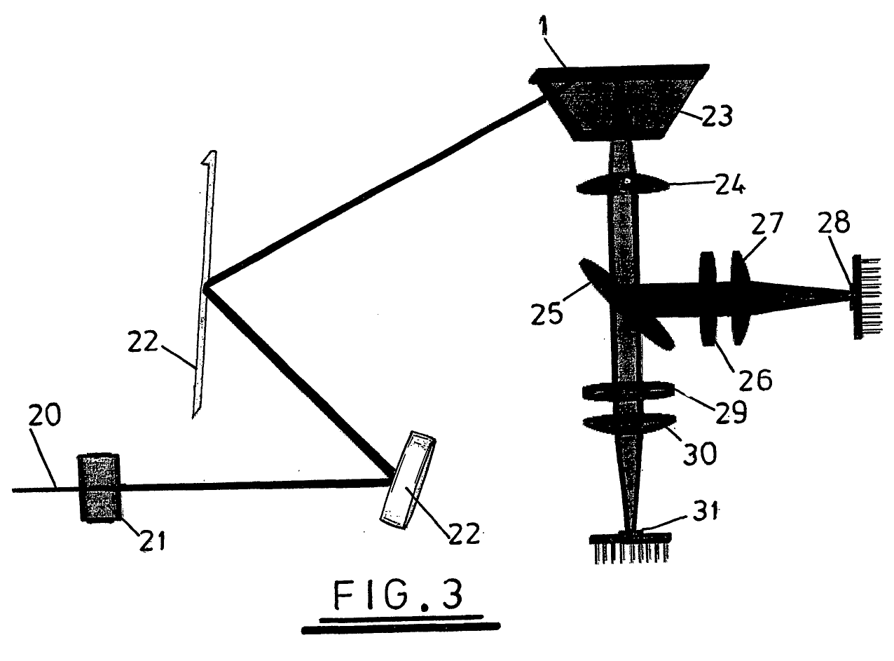

FIG. 3 is a schematic illustration of optical apparatus used as part of a flow cytometer which embodies the invention;

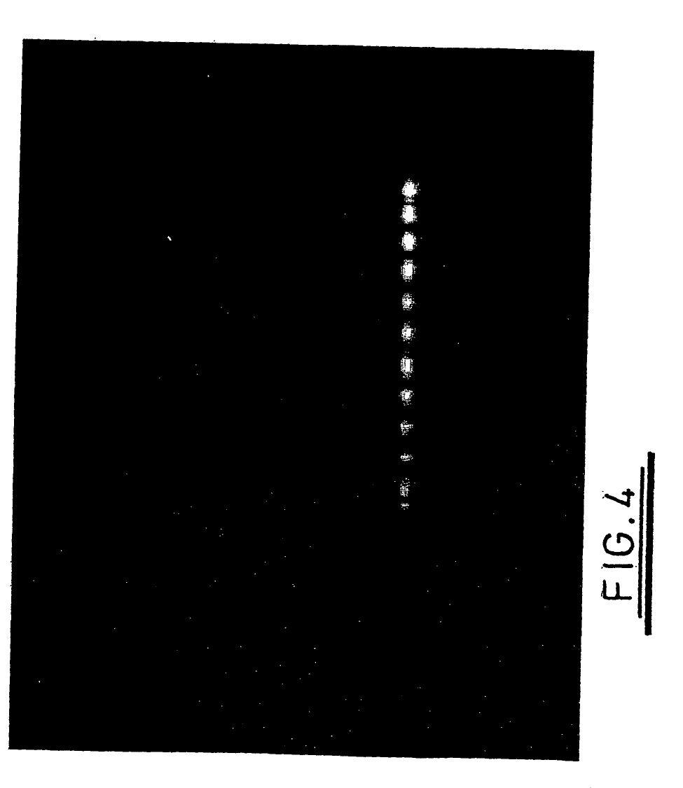

FIG. 4 is a transverse photograph showing fluorescence of sample fluid streams in a flow cytometer which embodies the invention;

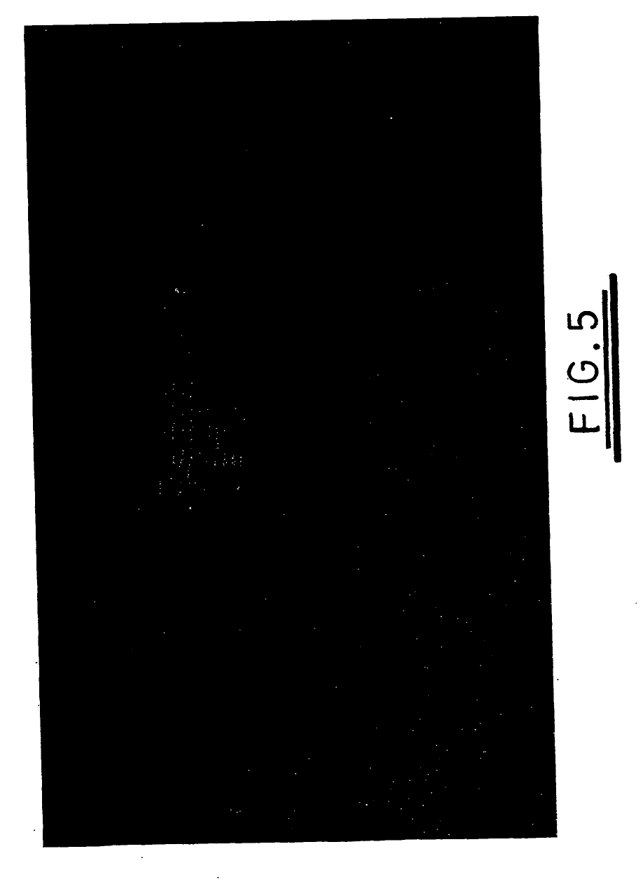

FIG. 5 is a plan photograph showing fluorescence of sample fluid streams in the flow cytometer shown in FIG. 4;

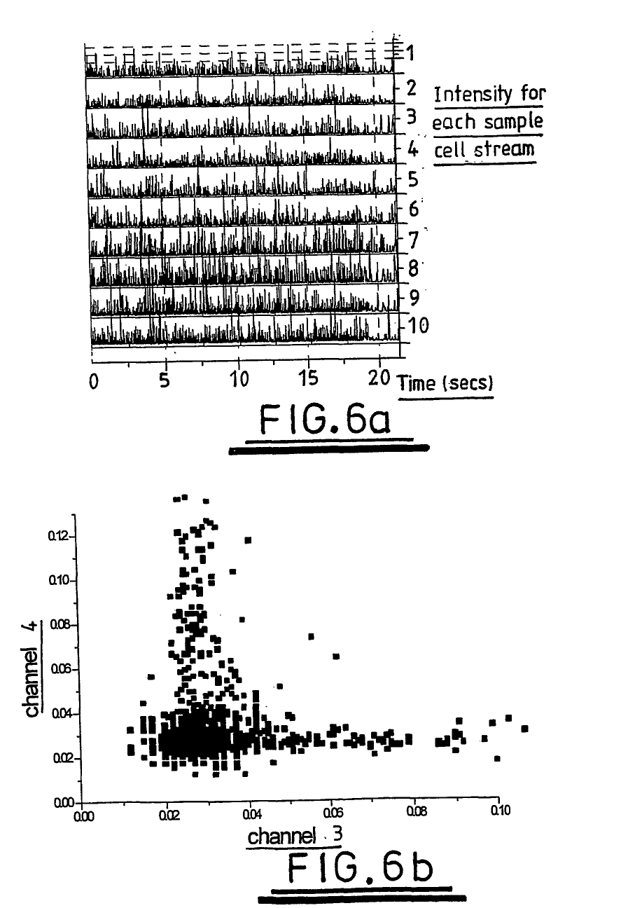

FIG. 6 is a pair of graphs illustrating detected fluorescence from fluid streams in the flow cytometer shown in FIGS. 5 and 6;

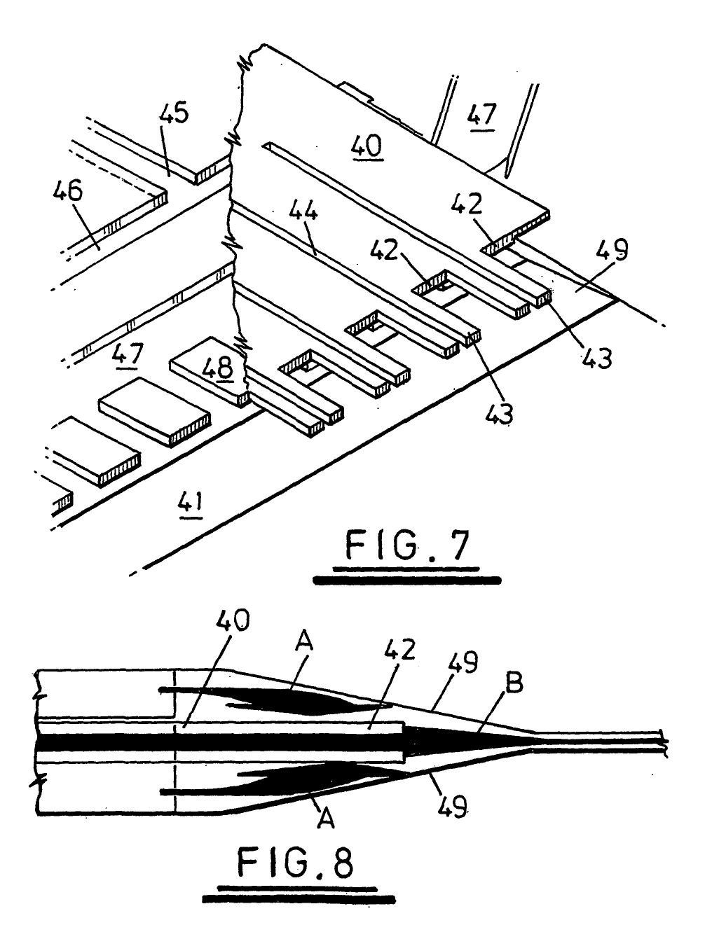

FIG. 7 is a schematic perspective view of a view cytometer which embodies the invention;

FIG. 8 is a side view of the flow cytometer of FIG. 7;

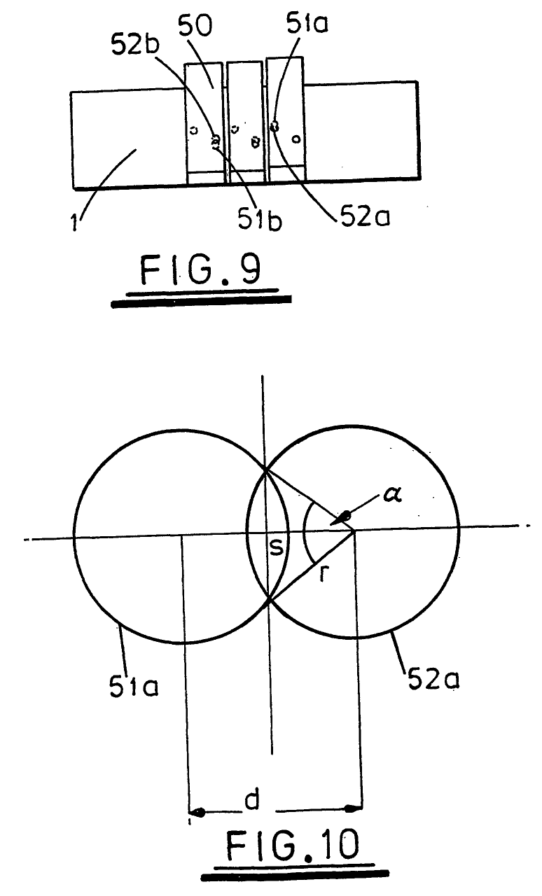

FIG. 9 is a plan view of a sorting mechanism of a flow cytometer which embodies the invention;

FIGS. 10 is a chart illustrating operation of the sorting mechanism shown in FIG. 9;

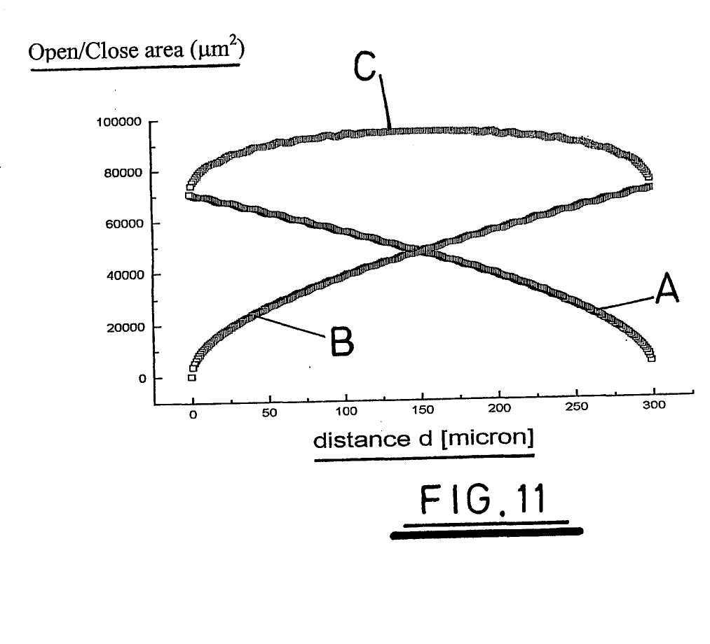

FIG. 11 is a graph illustrating operation of the sorting mechanism shown in FIG. 9;

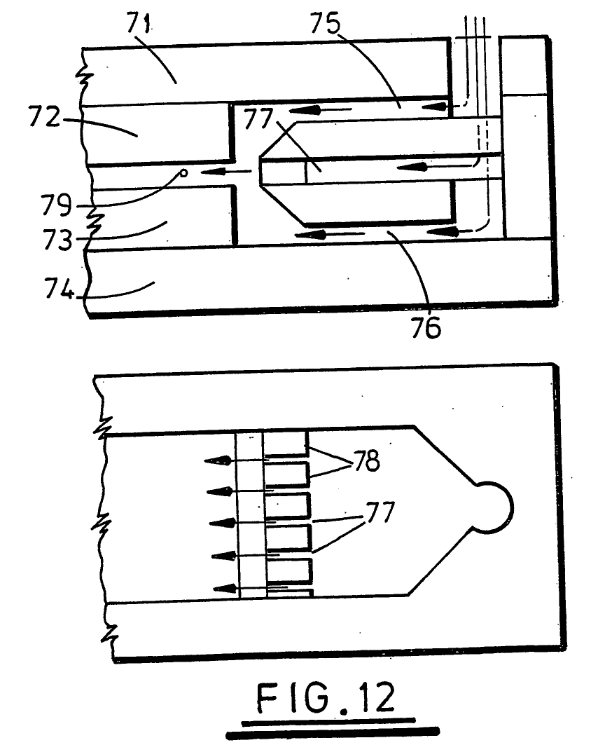

FIG. 12 is a schematic illustration of a flow cytometer which embodies the invention; and

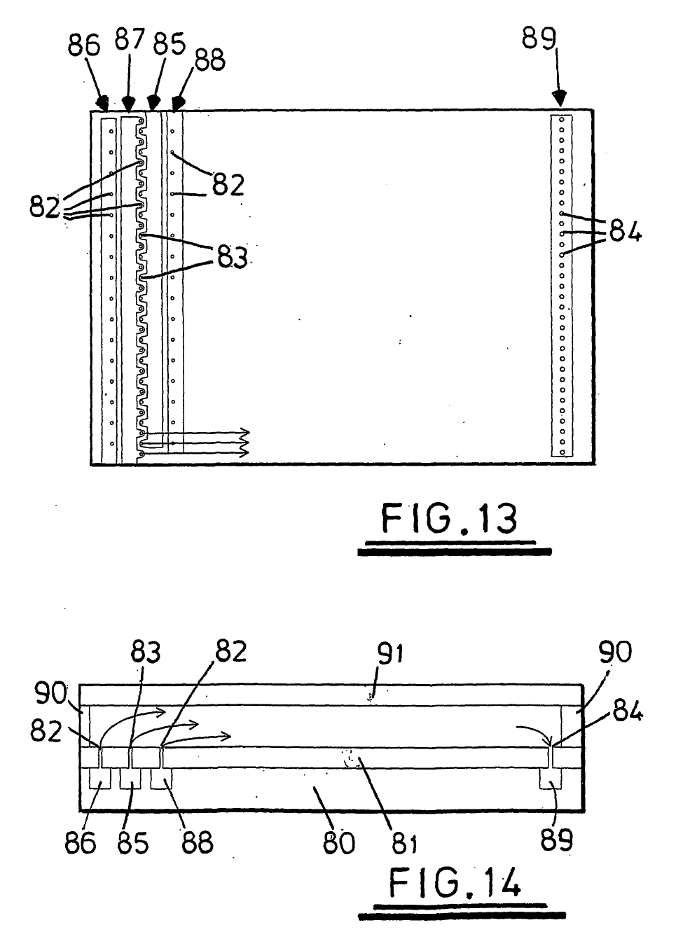

FIGS. 13 and 14 are schematic illustrations of a flow cytometer which embodies the invention.

Referring to FIG. 1, a flow cytometer comprises a disposable polymer chip 1 measuring 1 cm in width and 3 cm in length. The chip 1 has a flat upper surface. An inlet 2 is provided at an upstream end of the chip 1, to allow buffer fluid to flow onto the flat upper surface of the chip 1. The inlet 2 has a diameter of 2 mm.

Three separators 3 are provided downstream of the inlet 2. The separators 3 comprise walls which extend vertically from the surface of the chip 1, and are each provided with a taper at an upstream end. The shape of the separators 3 is such that buffer fluid which flows from the inlet 2 is separated into four laminar flows which flow past sides of the separators 3. The separators are 200 microns wide, and are spaced apart by 200 microns. The buffer fluid is phosphate buffered saline; it will be appreciated that any suitable buffer fluid may be used.

Sample inlets. 4 are provided immediately downstream of each of the separators 3, each sample inlet 4 having a diameter of 150 microns. The sample inlets 4 allow sample fluid to flow onto the flat upper surface of the chip 1. Sample fluid which flows onto the upper surface of the chip 1 flows as a laminar flow, and is constrained on either side by buffer fluid.

The buffer fluid provides hydrodynamic focussing of the sample fluid. The flow rate of the buffer fluid is selected to be sufficient that the sample fluid is focused to a stream of cells A aligned precisely in single file.

Light emitted by a laser is coupled to an optical mode 5 which crosses the chip 1, intersecting with each of the sample fluid streams A. Cells of interest in the sample fluid are provided with fluorescent labels. These fluorescent labels will emit fluorescent light when they pass through the optical mode 5 (the light coupled to the optical mode has a wavelength chosen to excite fluorescence of the labels). The presence of fluorescent light is detected separately for each sample using a 3-channel avalanche photodiode array (not shown), one channel being used per sample. The detection of a pulse of fluorescent light from a given sample stream A indicates that a fluorescent cell has passed through the optical mode 5. A photomultiplier array may be used in place of the avalanche photodiode array.

Downstream of the optical mode 5, the upper surface of the chip 1 is divided into three portions by a pair of walls 6. The walls 6 extend to a downstream end of the chip 1. Each sample fluid stream A passes, together with accompanying buffer fluid into a separate portion of the chip 1.

Each portion of the chip 1 is provided with two outlets 7a, 7b. One outlet, referred to hereafter as the collection outlet 7a, is intended to receive fluorescent labelled cells, and the other outlet, referred to hereafter as the waste outlet 7b, is intended to receive buffer fluid and sample fluid that does not contain fluorescent labelled cells.

A partition 8 is provided between each of the pairs of outlets 7a, 7b. Buffer fluid flows in a laminar flow into both of the outlets 7a, 7b. The diameter of the waste outlet 7b is marginally greater than the diameter of the collection outlet 7a, and consequently the flow of fluid drawn into the waste outlet 7b is slightly greater than the flow of fluid drawn into the collection outlet 7a. This slight disparity between flow rates causes the sample fluid stream A to be drawn into the waste outlet 7b.

When a fluorescent labelled cell passes through the optical mode 5 and is detected by the photodiode array, heat is applied to the buffer fluid immediately upstream of the collection outlet 7a using a laser beam 9. The viscosity of the buffer fluid is lowered by the applied heat, and the hydrodynamic resistance of the buffer fluid decreases. This increases the flow of buffer fluid drawn into the collection outlet 7a to a rate greater than the flow of buffer fluid into the waste outlet 7b, thereby causing the sample fluid stream A to flow into the collection outlet 7a The fluorescent labelled cell in the sample fluid thus passes into the collection outlet 7a. The laser beam 9 may be considered to be a cell sorting mechanism. In FIG. 1, fluorescent labelled cells have been detected in the first and third sample fluid streams A, and both of those fluid streams A are diverted using the laser beam 9 to the collection outlets 7a.

The laser beam 9 is generated with a time delay which is determined by the velocity of the sample fluid stream A, so that only fluorescent labelled cells are directed to the collection outlets 7a.

The required relative rates of fluid flow needed to provide sorting between the collection outlet 7a and the waste outlet 7b may be determined experimentally for a given cytometer configuration, by using an experimental arrangement which includes adjustable flow rates. Once the required flow rates have been determined a flow cytometer can be fabricated with means which provide the required flow rate without adjustment (for example restrictions could be introduced downstream of the outlets 7a, 7b).

To increase the change in viscosity caused by heating of the buffer fluid, a biocompatible additive such as glycerol can be added to the buffer fluid. The viscosity of glycerol decreases by a factor of 6 from 25 to 50° C., while water only decreases by a factor of less than 2. The glycerol will thus contribute significantly to the change of viscosity. Other suitable polymeric additives may be used to increase the change of viscosity.

The laser beam 9 is generated using an Indium Gallium Arsenide Phosphide (InGaAsP) laser diode with an emission wavelength of 1.55 μm (lasers of this type are commonly used in telecommunications applications). The absorption coefficient of water (a commonly used buffer fluid) at 1.55 μm is 10.6 cm⁻¹, as shown in FIG. 2. A 30 μm thick water layer will absorb around 18% of the energy of the laser beam 9. If no heat were to be lost from the water then, assuming a volume of 2.7×10⁻⁹ litres (300 by 300 by 30 microns) and a laser power of 10 mW, the water temperature will rise by 16 degrees centigrade in 0.1 seconds. In practice, heat is lost very quickly from the water by diffusion which occurs over a millisecond time scale. This means that around 100 mW is required to heat the water by around 16 degrees centigrade. The change in viscosity caused by an temperature increase from 20 C (102×10⁻⁶ kg s/m² (Poise)) to 40 C (66.5×10⁻⁶ Poise) will cause a significant drop in the hydrodynamic resistance of the water.

A bottleneck may be provided in the region to be heated by the laser, the bottleneck having a diameter which corresponds to that of the laser beam (typically around 200 μM). This helps to ensure that the laser beam heats the water efficiently.

The polymer used to construct the chip 1 is transparent to light at 1.55 μm, so that the second laser beam 9 can be directed onto the buffer fluid without being absorbed by the chip 1. A preferred polymer is PMMA, because it provides very low absorption of light at 1.55 μm.

Light at 1.48 μm provides very efficient heating of water because it lies closer to the top of the absorption peak shown in FIG. 2. For this reason a laser which generates 1.48 μm light may be used to heat the water (the laser may for example be a diode laser). The chip 1 is constructed from a polymer which is transparent to light at 1.48 μm, for example PMMA.

A suitable dye can be added to the buffer stream to provide strong absorption of laser light and thereby heat the buffer fluid. For example, 1,3-bis[4-(dimethylamino)phenyl]-2,4-dihydrocyclobutenediylium dihydroxide bis(inner salt) can be added in low concentration to the buffer stream to absorb visible light around 635 nm, as it has a very high extinction coefficient of ˜250000 1 mol⁻¹ cm⁻¹. A laser which emits light at around 635m would be used to heat the fluid.

The buffer fluid may be heated in other ways. For example, heating of the buffer stream can also be effected using DC or AC electrolytic heating. To do this, two electrodes are placed on either side of a channel region to be heated. Referring to FIG. 1 this would be upstream of the collection outlets 7a, at the positions where the laser beams 9 are shown (the laser beams are not required for electrolytic heating). The electrodes are placed either across the width or depth of the channels. The electrodes can be metallic, such as copper or gold or platinum plated copper, or made from a polymer loaded with a conductor such as carbon fibre or nickel-plated carbon fibre. Heating is carried out by passing a current through the buffer stream by applying a suitable voltage to the electrodes. The applied voltage is preferably alternated at a suitable frequency to avoid significant electrolysis and gas bubble generation at the electrodes.

Fluorescence from each sample fluid stream may be imaged by an imaging spectrograph (not shown) onto a two-dimensional detector array to detect different wavelengths of fluorescence emission. The imaging spectrograph may be a flat-field concave holographic grating and the detector array a multi-anode photomultiplier.

The chip 1 is provided with an upper layer (not shown) which is arranged such that the sample fluid stream and the buffer fluid are sandwiched between the upper surface of the chip 1 and a lower surface of the upper layer. The refractive index of the chip 1 and the upper layer is 1.6, whereas the refractive index of the buffer fluid and the refractive index of the sample fluid are both approximately 1.33.

A conventional optical mode is supported by a high refractive index layer sandwiched between low refractive index layers. A refractive index profile comprising a low refractive index region sandwiched between high refractive index layers is not capable of supporting a conventional optical mode. Thus, the optical mode S directed across the flow cytometer cannot be a conventional optical mode.

A recently discovered optical mode, known as a leaky waveguide mode, is supported by a low refractive index region sandwiched between high refractive index layers. The leaky waveguide mode is confined by high Fresnel reflection which occurs at glancing angles of incidence at the interfaces between the low refractive index region and the high refractive index layers. One leaky waveguide mode, known as a Light Condenser (LC) mode has the property that its optical leakage is very low, which means that it is able to propagate several centimetres without suffering significant loss. The LC mode is excited by directing light into a low refractive index region sandwiched between high refractive index regions such that light within the low refractive index region will strike the high refractive index regions at angle greater than the critical angle. The light will be reflected from the high refractive index regions and will propagate as a mode along the low refractive index region. The LC mode is described in Chapter 5 of Internal Reflection Spectroscopy, N. J Harrick, published by John Wiley, New York, 1967. The LC mode is also described in PCT/GB99/00399.

The inventors have realised that the LC mode is suitable for providing illumination of sample fluid streams in a flow cytometer of the type illustrated in FIG. 1. The optical mode 5 shown in FIG. 1 is a LC mode which is supported by the refractive index profile provided by the buffer fluid, the chip 1 and the upper layer. The LC mode will not encounter significant refractive index steps when crossing the flow cytometer, and will not suffer significant loss. This means that each of the sample fluid streams A is illuminated by a high intensity mode. A high intensity mode is preferred because it will excite fluorescence in each fluorescent labelled cell. Where a low intensity mode is used, fluorescence will not be excited in each fluorescent labelled cell. Those labelled cells which are not excited will be directed to the waste outlet 7b and will be lost.

The LC mode may be directed across the flow cytometer, or alternatively may directed along the flow cytometer.

Since the LC mode does not suffer significant loss, it may be used to excite fluorescence in a large number of parallel sample fluid streams (10 or more). The sample fluid streams should preferably be separated by buffer fluid provided as a laminar flow.

Different orders of LC mode may be excited. A single order LC mode has a generally Gaussian type cross-section, whereas higher order LC modes are more square in cross section. A higher order LC mode will provide more even illumination (in terms of cross-section) across a region of interest, but will suffer from the disadvantage that it will lose intensity and change shape more quickly as it propagates.

The chip 1 may, instead of having an upper layer, have no upper layer and simply be exposed to the atmosphere (this may have a detrimental effect on the laminar flow of the buffer fluid). Where this is done, an optical mode directed across the chip 1 will be confined at a lower boundary of the buffer fluid by light condenser reflection, and will be confined at an upper boundary of the buffer fluid by conventional total internal reflection. For ease of terminology an optical mode of this type will also be referred to as a light condenser mode.

An apparatus suitable for launching a LC mode into a flow cytometer, and detecting fluorescent light emitted by a sample fluid stream is illustrated in FIG. 3. A laser beam 20 is generated by a solid state laser operated at 473 nm in a nominal TEM₀₀ mode (Laser 2000). The power of the laser beam is fixed at 10 mW. The laser beam 20 is passed through a rod lens 21 which expands the beam 20 in a transverse direction. The beam 20 is steered using a pair of steering mirrors 22 into a Dove prism 23 (F5, refractive index 1.6). The Dove prism 23 is used to prism-couple the laser beam 20 into a flow cytometer 1 of the type illustrated in FIG. 1, where it is launched as a LC mode which passes through sample fluid streams flowing across the flow cytometer. Fluorescent light emitted by fluorescent labelled cells, and scattered light, is collected using an aspheric lens 24 (f=8 mm), and passes to a dichroic beamsplitter 25. The dichroic properties of the beamsplitter 25 are chosen such that fluorescent light is reflected by the dichroic beamsplitter 25 and scattered light is transmitted by the dichroic beamsplitter 25. The fluorescent light passes through an interference filter 26, and is focused by a lens 27, onto a first avalanche photodiode array 28. The scattered light passes through an interference filter 29, and is focussed by a lens 30 onto a second avalanche photodiode array 31. Detected scattered light is useful because it provides an indication of the size of a particle. Knowledge of particle size may be used to normalise detected fluorescent light.

The light condenser mode may propagate along the direction of travel of the sample fluid streams.

Scattered light that is collected in a cone which lies in a direction the orthogonal to an excitation mode, as described above, is often referred to a side scattering (SSC). Side scattering provides an indication of particle size, but suffers from the disadvantage that it may be affected by surface and internal structures.

Light scattering over small angles relative to the direction of propagation of an excitation mode is often referred to as forward scattering (FSC). Forward scattering also provides an indication of particle size, and is less likely to be affected by surface and internal structures.

Forward scattering produces generally a strong signal, which can be detected by using a photodiode, whereas the signal produced by side scattering requires is typically significantly weaker. This is particularly the case when the refractive index of the sample particles is close to the refractive index of the buffer fluid. A photomultiplier tube or avalanche photodiode may be required to detect side scattering.

Side scattering and forward scattering may both be measured using light condenser mode excitation. For example, a light condenser mode may be directed across the cytometer in a direction perpendicular to the direction of sample propagation, and side scattering may be detected using a 16 channel photo-multiplier tube. In order to detect forward scattering a detector must be positioned at small angle relative to the direction of propagation of the mode.

In some instances it may be the case that the intensity of scattering provided from light condenser mode excitation is not sufficiently strong to provide a useful measurement, particularly in the case of side scattering. For this reason it may be preferred to perform scattering measurements by focussing a laser beam onto the cytometer, without exciting a light condenser or other mode. Since the cytometer carries several parallel samples the laser beam is separated into multiple beams using a diffracting beam splitter, and the separated beams are focussed onto sample streams in the cytometer. For forward scattering separated focussed laser beams with a spot size of around 20 μm may be used, with detectors located between 3 and 8 degrees away from the direction of propagation of the beam (typically the laser beams are directed through the cytometer).

The flow cytometer chip illustrated m FIG. 1 was fabricated as follows. Dry film negative photoresist is laminated onto polymer substrates (preferably polycarbonate sheets between 0.75 and 2 mm thick) with a hot roll laminator. Photomasks representing a number of flow cytometer chips arranged in an array are located over the photoresist, and are illuminated by UV light. The photoresist is developed in a 1% potassium carbonate solution to remove non-crosslinked resist (i.e. any photoresist that has not been exposed to UV light), thereby providing an array of patterned chips. An unlaminated polycarbonate sheet with predrilled injection/access holes is thermally bonded onto the patterned chip. Finally, individual chips are cut from the array of chips using a guillotine.

The above method of fabrication was also used to make a 10 channel flow cytometer chip dimensioned to match a commercially available avalanche photodiode array, which has 10 channels with a pixel width of 200 μm and pitch of 400 μm (fabricated by Silicon Sensors, of Berlin, Germany). The thickness of the resist used to fabricate the flow cytometer chip is 30 μm. The sample fluid streams are spaced 400 μm apart. Separators are 200 μm wide each, leaving 200 μm channels for buffer fluid flows. Sample inlet holes are provided with a diameter of 150 μm. The sample fluid is directed to the sample inlet holes via a milled Perspex block glued to an underside of the chip. The Perspex block houses 2 mm PVC tubing. Buffer fluid inlets are also connected to 2 mm PVC tubing, as are waste outlets.

FIGS. 4 shows illumination of fluorescein stained latex beads passing in fluid streams across the surface of a 10 sample fluid stream flow cytometer chip constructed as described above. The beads are illuminated using a LC mode which traverses the flow cytometer. FIG. 4 was recorded using an avalanche diode array. FIGS. 4 demonstrates that the intensity of the LC mode remains sufficiently high that fluorescence is excited in the tenth fluid stream of the flow cytometer (the left hand fluid stream in FIG. 4).

FIG. 5 also shows illumination of fluorescein stained latex beads passing in fluid streams across the surface of a 10 sample fluid stream flow cytometer chip. In FIG. 5 an LC mode propagates along the direction of flow of the fluid streams rather than traversing the fluid streams. Coupling to the flow cytometer chip was provided using the apparatus shown in FIG. 3. A flow cytometer chip which is illuminated as shown in FIG. 5 may be used to monitor dynamic effects, for example a reaction process which occurs during passage of a sample along the chip. A two-dimensional array of sensors may be used to monitor samples which are illuminated along their direction of flow. A disadvantage of directing an LC mode along the direction of flow is that the intensity of the illumination is less than that which would be provided by transverse illumination, and the amount of fluorescence that will be excited is correspondingly reduced.

FIG. 6a shows the intensity of fluorescent light detected for the 10 channel flow cytometer chip by the avalanche photodiode array over a 20 second period. Each peak corresponds to the passage of a fluorescein stained latex bead through the LC mode. Cross-talk of the light detected for adjacent sample fluid flows is low, as illustrated by FIG. 6b.

FIG. 7 illustrates an alternative configuration of sample fluid inlet and buffer fluid inlet. The sample fluid inlet comprises a 100 μm thick laser cut sheet stainless steel sheet 40, which is bonded onto an injection moulded polymer layer 41. Part of the steel sheet 40 is cut away in FIG. 7 (for illustration purposes only) to expose the polymer layer 41. A series of notches 42 are cut into one end of the sheet 40, thereby providing separated extending surfaces 43. A 100 μm wide channel 44 is cut along a centre of each extending surface 43 and extends back towards a rear end of the sheet 40. Sample fluid is provided by a channel 45 in the polymer layer 41. The sample fluid is distributed by a transverse channel 46 to the channels 44 which are in fluid communication therewith.

A second transverse channel 47 is provided in the polymer layer 41 to distribute buffer fluid. Rectangular spacers 48 are provided in the transverse buffer channel 47 which act to separate the buffer flow into spaced apart streams. An upper surface of the polymer layer 41 is provided with an upward taper 49. The effect of the taper 49 and the spacing of the buffer streams is such that the buffer fluid flows onto and around the extending steel surfaces 43, providing hydrodynamic focussing of the sample fluid when it leaves the channels 44 of the extending steel surfaces 43.

A second polymer layer (not shown in FIG. 7) with the same configuration as the first polymer layer 41 is bonded to an upper surface of the steel sheet 40.

FIG. 8 illustrates the flow of buffer fluid and sample fluid. Buffer fluid flows around the spacers provided in the polymer layers. The buffer fluid flows around the extending surfaces 42 of the steel sheet 40, as indicated by the arrows A, to provide a laminar flow along the extending surfaces 42. The taper 49 of the polymer layers causes the flow of the buffer fluid A to accelerate as it passes along the extending surfaces 42. Sample fluid as indicated by the arrow B leaving the channels 43 at the ends of the extending surfaces 42 is hydrodynamically focussed in two dimensions by the buffer fluid A as it flows around the extending surfaces 42. This is represented in FIG. 8 by tapering of the sample fluid B. The sample fluid B is focussed into a series of hydrodynamically focussed sample fluid streams flowing in a parallel direction within buffer fluid A (the buffer fluid A has a laminar flow).

Light is coupled across the sample fluid streams as a light condenser optical mode.

A flow cytometer similar to that illustrated in FIGS. 7 and 8 may be fabricated using two sheets of steel instead of a single sheet of steel. Where this is done, sample channels are etched partway into the two sheets and the sheets are subsequently aligned to provide sample channels.

Flow cytometers include a sorting mechanism, which directs fluorescent labelled sample cells to a collection outlet and directs the remainder of a sample to a waste outlet. As described above, the flow cytometer illustrated in FIG. 1 uses a laser beam to heat buffer fluid, thereby reducing its viscosity and altering the flow direction of a sample fluid stream so that it flows into a collection outlet. An alternative sorting mechanism is illustrated in FIG. 9. The sorting mechanism illustrated in FIG. 9 comprises a stainless steel plate 50 which is located over a flat surface of a flow cytometer chip 1 (shown in part). The stainless steel plate is provided with a pair of 300 μm diameter holes, collection hole 51a, and waste hole 51b. The collection and waste holes 51a, 51b are deliberately misaligned with respect to collection and waste outlets 52a, 52b of the flow cytometer chip 1. The collection and waste outlets 52a, 52b are also 300 μm in diameter.

When the steel plate 50 is in a first position, the collection hole 51a is located directly over the collection outlet 52a, and the waste hole 51b overlaps only partially with the waste outlet 52b (this is illustrated by the left hand and middle steel plates shown in FIG. 9). When the steel plate 50 is in this position, the flow of buffer fluid into the collection outlet 52a is greater than the flow of buffer fluid into the waste outlet 52b, and a sample fluid stream will be directed towards the collection outlet 52a.

When the steel plate 50 is in a second position, the collection outlet hole 51a coincides only partially with the collection outlet 52a, and the waste outlet hole 51b is located directly over the waste outlet 52b (this is illustrated by the right hand steel plate shown in FIG. 9). When the steel plate 50 is in this position, the flow of buffer fluid into the collection outlet 52a is less than the flow of buffer fluid into the waste outlet 52b, and a sample fluid stream will be directed towards the waste outlet 52b.

The plates 50 are moved between the first and second positions using a piezo-electric translator which is controlled in response to the detection of fluorescent cells passing through a light condenser mode coupled across sample fluid streams upstream of the sorting mechanism.

FIG. 10 illustrates the manner in which the area of overlap between an outlet hole 51a and an outlet 52a varies during movement of the steel plate. The area of overlap is illustrated in FIG. 10 as a segment ‘S’. The area of the segment S varies as follows:

S=r²(πα/(180−sin α))/2

FIG. 11 illustrates the area of overlap of two pairs of circles 30 μm in diameter. A first pair of circles A begins with a full overlap (70000 μm²), and a second pair of circles B begins with a zero overlap. The rate of change of ‘d’ is identical for both pairs of circles. It can be seen that for close to zero, the rate of change of overlap is not constant, i.e. the lines in FIG. 11 are not straight. However, for overlaps greater than 75 μm, the rate of change is substantially constant. This is illustrated clearly by the total area of overlap for both pairs of circles, which is indicated by the line C.

The collection and waste holes 51a, 51b and collection and waste outlets 52a, 52b are staggered so that the area of overlap of a hole and outlet is never less than 60 μm. This ensures that the total area of overlap between the holes and outlets is substantially constant irrespective of the position of the steel plate 50, and that the total flow rate of sample fluid and buffer fluid in the cytometer is constant.

The sorting mechanism may be fabricated separately from the rest of the flow cytometer chip, and subsequently bonded to the flow cytometer chip.

Another sorting mechanism which may be used in conjunction with any of the flow cytometer chips described above is a mechanical valve located downstream of collection and waste outlets, arranged to switch flow of a sample fluid stream between the collection and waste outlets by adjusting the flow through each. The mechanical valve may be a piezo-electric switch.

Other sorting mechanisms based upon heating the buffer fluid using laser heating, or electrical heating are described further above. Other suitable sorting mechanisms will be apparent to those skilled in the art.

A flow cytometer chip constructed using injection moulding is shown in FIG. 12. The chip is constructed in four separate layers 71-74 each 2 mm thick which are bonded together. The chip is provided with upper and lower buffer inlets 75,76 which extend across a central 13 mm section of the chip. The chip is provided with a series of sample inlets 77, each inlet being 100 μm across and being spaced 900 μm away from adjacent inlets by spacers 78. A light condenser optical mode 79 propagates across the chip. The chip includes a sorting mechanism (not shown).

Any of the flow cytometer chips described above may be fabricated cheaply, allowing them to be disposable (i.e. discarded after a single use). This is advantageous because it avoids the possibility of a flow cytometer measurement being contaminated by residual traces of a sample used in an earlier flow cytometer measurement.

A further flow cytometer chip which embodies the invention is shown in FIGS. 13 and 14. Referring to FIGS. 13 and 14, a 16-channel flow cytometer is fabricated in poly-methylmethacrylate by direct computer numerically controlled (CNC) machining. The chip consists of four layers. A substrate 80 is 6 mm thick, 78 mm long and 78 mm wide. A 2 mm thick intermediate layer 81 is located on the substrate. The intermediate layer 81 contains drilled buffer inlets 82 of 250 μm diameter, drilled sample inlets 83 of 150 μm diameter and waste outlets 84 of 250 μm diameter. The substrate 80 is provided with a sample distribution channel 85 which feeds the sample to the sample inlets 83. Further channels 86-88 feed buffer fluid to the buffer inlets 82. A waste collection channel 89 is connected to the waste outlets 84. The channels 85-89 have a cross sectional area of 1 mm², and are spaced on a 1 mm pitch.

A layer of 30 μm thick dry-film photoresist is laminated on top of the intermediate layer and the rectangular area over which the sample and buffer fluid flows is defined photolithographically in the photoresist. The remaining photoresist 90 is visible at either end of the cytometer in FIG. 14, but is not shown in FIG. 13 (the thickness of the photoresist is not shown to scale). A 4 mm thick PMMA cover layer 91 is bonded to the remaining photoresist 90 to seal the area defined by the photoresist. The rectangular area over which the sample and buffer fluid flows is 24 mm long by 17 mm wide.

In use, sample and buffer fluid flows from the inlets 82, 83 and into the area defined by the photoresist Referring to FIG. 13, lateral focussing of the sample is provided by buffer fluid which flows on either side of the sample. Both the buffer fluid and the sample flow as a laminar flow. Referring to FIG. 14, vertical focussing of the sample is provided by buffer fluid which flows above and below the sample. Again, the buffer fluid and the sample fluid flow as a laminar flow. The effect of the buffer fluid which is introduced downstream of the sample fluid (FIG. 14) is to push the sample fluid flow vertically upwards as shown This is advantageous because the sample will overlap with the centre of any optical mode which is coupled to the cytometer.

The cytometer shown in FIGS. 13 and 14 does not include any cell sorting mechanism. However, it will be appreciated that any suitable cell sorting mechanism including those described further above, may be added to the cytometer.

1. An identification apparatus comprising a chamber having one or more buffer fluid inlets and a plurality of sample fluid inlets, the chamber being configured such that the buffer fluid focuses the sample fluid into sample fluid streams, the sample fluid streams and buffer fluid being contained in a single laminar flow, wherein the apparatus is provided with means for directing optical excitation simultaneously at a plurality of the sample fluid streams to allow parallel fluorescence and/or scattering measurements.

2. An identification apparatus according to claim 1, wherein the chamber supports an optical mode centred on the single laminar flow, which passes through the sample fluid streams, thereby providing the optical excitation.

3. An identification apparatus according to claim 2, wherein the optical mode propagates across the single laminar flow in a direction substantially perpendicular to the direction of flow.

4. An identification apparatus according to claim 3, wherein the optical mode propagates along the single laminar flow in a direction substantially parallel to the direction of flow.

5. An identification apparatus according to any of claims 2 to 4, wherein the chamber is defined by upper and lower walls having refractive indices greater than the refractive index of the buffer fluid and sample fluid, and the optical mode is a leaky waveguide mode.

6. An identification apparatus according to claim 5, wherein the upper and lower walls are constructed from a polymer.

7. An identification apparatus according to claim 5 or 6, wherein the leaky waveguide mode is a light condenser mode.

8. An identification apparatus according to claim 1, wherein the optical excitation directing means comprises a diffracting beam splitter for separating a laser beam into a plurality of beams, and means for directing the plurality of beams at the sample fluid streams

9. An identification apparatus according to any preceding claim, wherein the sample fluid inlets are located downstream of at least some of the one or more buffer fluid inlets

10. An identification apparatus according to any preceding claim, wherein the sample fluid contains fluorescent labelled cells, the optical excitation has a wavelength suitable for exciting fluorescence of the fluorescent labelled cells, and the apparatus is provided with a detector to detect fluorescence of the fluorescent labelled cells.

11. An identification apparatus according to claim 10, wherein the detector is an array, each channel of the array detecting fluorescence from a different sample fluid stream.

12. An identification apparatus according to claim 11, wherein the apparatus is provided with an imaging spectrograph arranged to image fluorescence from sample fluid streams, and the array is a two-dimensional array arranged to detect different wavelengths of fluorescence emission.

13. An identification apparatus according to any preceding claim, wherein the apparatus is provided with a collection outlet and a waste outlet, and a sample stream is directed to the collection outlet in response to the detection of a fluorescent labelled cell in that sample fluid stream.

14. An identification apparatus according to claim 13, wherein a plate is located over the collection outlet and waste outlet, the plate having a collection opening and a waste opening, wherein when the plate is in a waste position the waste opening and the waste outlet are substantially aligned and the collection opening and the collection outlet are substantially misaligned to allow a lesser amount of fluid to pass into the collection outlet, and when the plate is in a collection position the collection opening and collection outlet are substantially aligned and the waste opening and waste outlet are substantially misaligned.

15. An identification apparatus according to claim 14, wherein the collection opening and the waste opening are arranged such that the total flow of fluid into the collection outlet and waste outlet is substantially unchanged by movement of the plate from the collection position to the waste position.

16. An identification apparatus according to claim 15, wherein the openings and the holes are substantially circular, and are arranged to have an overlapping area of at least 20% when they are substantially misaligned.

17. An identification apparatus according to any preceding claim, wherein the chamber is provided with a plurality of separator walls having tapered upstream ends, the walls being located downstream of the buffer fluid inlet, and being arranged to separate the buffer fluid into a plurality of flows.

18. An identification apparatus according to claim 17, wherein a sample inlet is located substantially immediately downstream of each separator wall, such that the flow of buffer fluid around the sample inlet hydrodynamically focuses the sample fluid into a sample fluid stream.

19. An identification apparatus according to any of claims 1 to 16, wherein the sample fluid inlets and at least some of the buffer fluid inlets open onto a substantially planar surface, the inlet openings lying in substantially the same plane.

20. An identification apparatus according to claim 19, wherein at least some of the buffer fluid inlets are located downstream of the sample fluid inlets, thereby providing a layer of buffer fluid between the sample fluid streams and the substantially planar surface.

21. An identification apparatus according to any of claims 1 to 16, wherein the sample fluid inlets comprise fingers extending in a downstream direction, each finger being provided with a channel which carries the sample fluid to an opening at a downstream end of the finger.

22. An identification apparatus according to claim 21, wherein each finger is spaced way from upper and lower surfaces of the chamber, such that the buffer fluid flows along sides of the finger, and hydrodynamically focuses sample fluid emitted from the finger into a sample fluid stream.

23. An identification apparatus according to claim 21 or claim 22, wherein the fingers are constructed from stainless steel.

24. An identification apparatus according to any of claims 1 to 20, wherein the identification apparatus is fabricated by injection moulding a plurality of layers and then bonding those layers together.

25. An identification apparatus comprising a chamber having one or more buffer fluid inlets and one or more sample fluid inlets, the chamber being configured such that the buffer fluid focuses the sample fluid into one or more sample fluid streams, wherein the apparatus is provided with a collection outlet and a waste outlet in separated portions of the chamber, and means for directing the sample fluid stream towards the collection outlet as required by directly heating buffer fluid in a portion of the chamber which is located upstream of the collection outlet so as to modify the fluid properties of the buffer fluid.

26. An identification apparatus according to claim 25, wherein the buffer fluid is directly heated using a laser.

27. An identification apparatus according to claim 26, wherein the buffer fluid contains a dye to provide enhanced absorption of the laser.

28. An identification apparatus according to claim 25, wherein the chamber is provided with electrodes which are in contact with the buffer fluid, and the buffer fluid is directly heated by passing a current between the electrodes via the buffer fluid.

29. An identification apparatus according to claim 28, wherein the current passed between the electrodes is alternated to avoid significant electrolysis and gas bubble generation at the electrodes.

30. An identification apparatus according to any of claims 25 to 29, wherein the buffer fluid includes a viscosity modifying substance to provide enhanced changes of the viscosity of the buffer fluid in relation to changes of fluid temperature.

31. An identification apparatus according to claim 30, wherein the viscosity modifying substance is glycerol or a polymer.

32. An identification apparatus according to any of claims 25 to 31, in combination with any of claims 1 to 24.

33. An identification apparatus according to any preceding claim substantially as hereinbefore described with reference to the accompanying figures.
